(19) United States
(12) Patent Application Publication
(10) Pub. No.: US 2005/0025822 A1
(43) Pub. Date: Feb. 3, 2005

(54) Method of pulmonary administration of an agent

(76) Inventors: Frances M.P. Wong, Redwood City, CA (US); Anuk Das, Wayne, PA (US); Jonathan Seideman, New York, NY (US); Luke Guo, Lafayette, CA (US); Anthony Huang, Saratoga, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/856,559

(22) Filed: May 27, 2004

Related U.S. Application Data
(60) Provisional application No. 60/475,080, filed on May 30, 2003.

(51) Int. Cl. A61K 9/127; C12N 15/88

(52) U.S. Cl. 424/450; 435/458

(57) ABSTRACT

A method for administering a therapeutic or diagnostic agent to a subject is described. The method includes providing a suspension of liposomes comprised of one or more of vesicle-forming lipids selected from (i) a vesicle-forming lipid derivatized with a hydrophilic polymer and (ii) a neutral lipopolymer, said liposomes being associated with said therapeutic or diagnostic agent, forming an aerosol of said liposome suspension; and administering the aerosol to the subject by inhalation. The liposome formulation delivers intact liposomal particles to the respiratory tract of said subject to form a depot of therapeutic agent therein with no observable provocation of an immune response, as measured by neutrophil or macrophage cell count in the lung after administration.

This application claims the benefit of Provisional Application No. 60/475,080, filed May 30, 2003.

### FIELD OF THE INVENTION

The present invention relates to a method for delivering a therapeutic or diagnostic agent to the respiratory tract of a subject. More specifically, the invention relates to a method of delivering such an agent associated with liposome particles with no provocation of an immune response.

### BACKGROUND OF THE INVENTION

Delivery of drugs via inhalation is a convenient and feasible route of administration with the advantage of directed delivery and minimizing the toxicity of many therapeutic agents. This method of administration can be applied to a number of indications including inflammatory and fibrotic pulmonary diseases, respiratory tract infections, lung cancers and cystic fibrosis. Furthermore, the lung can also be used as a convenient portal of administration for small and macro-molecules for systemic applications.

Inhalation appears to have many advantages associated with delivery. However, the portal to administration, the lung, is sensitive to irritants. Therapeutic agents, both small molecules and macromolecules, and diagnostic agents can cause significant irritation and/or toxicity when administered to lung tissue. Immune reactions that are initiated upon administration of foreign materials to lung tissue can immediately impact lung function and initiate chronic events. While there are a host of mechanisms in the lung which are used to remove molecules that induce immunogenicity, such as the mucociliary escalator, cellular immune responses, and complement activation, these mechanisms are also associated with immune stimulation. The long-term effects of lung inflammation mediated by activation of macrophages and cytokines is unknown, but can include pulmonary fibrosis and mucus hypersecretion leading to compromised lung function and persistent bronchoconstriction (Zhang,-H. J. et al., Immunology 101(4):501, (2000)).

Activation of and phagocytosis by alveolar macrophages is a first step in the inflammatory process upon administration of an irritant directly to the lung. This can lead to a cascade of immune events leading to both innate and acquired immunity. One of the first consequences of macrophage activation is the production of cytokines and chemokines , such as TNFα, IL-1β, IL-6, MCP-1, the stimulation of adhesion molecules as well as secretion of NO and reactive oxygen species, among others (de Haan, A. et aL., Immunology, 89(4): 488 (1996); Lentsch, A. B., et a., Am. J. Respir. Cell Mol Biol., 20(4):692 (1999)). These effector molecules recruit and stimulate other immune cells, mainly neutrophils, into the lung. The recruitment and activation of macrophages and neutrophils can cause tissue damage as a result of cell byproduct release and vasodilation (Phan, S. H. et al., Exp. Lung Res., 18(1):29 (1992)).

A delivery system that does not induce inflammatory or immune effects upon inhalation remains to be identified. Ideally, such a delivery system would additionally reduce or eliminate inherent toxicities of therapeutic agents.

One approach to pulmonary delivery has been to entrap therapeutic agents in liposomes (see, for example, U.S. Pat. Nos. 5,043,165; 5,958,378; 6,090,407; 6,103,746; 6,346,223; WO 86/06959). The liposomes are aerosolized for delivery to the lung. However, there remains a need in the art for a liposomal formulation that can be delivered to the lungs and which does not provoke an immune response, yet provides a depot reservoir of drug for a sustained release.

### SUMMARY OF THE INVENTION

Accordingly, it is an object of the invention to provide a method of administering a therapeutic or diagnostic agent to a subject via inhalation of the agent in the form of an aerosolized liposomal carrier.

In one aspect, the invention includes a method for administering a therapeutic or diagnostic agent to a subject, comprising providing a suspension of liposomes comprised of one or more of vesicle-forming lipids selected from (i) a vesicle-forming lipid derivatized with a hydrophilic polymer and (ii) a neutral lipopolymer, the liposomes being associated with said therapeutic or diagnostic agent; forming an aerosol of said liposome suspension; and administering the aerosol to the subject by inhalation, whereby said administering delivers intact liposomal particles to the respiratory tract of the subject to form a depot of therapeutic agent therein with no observable provocation of an immune response as measured by neutrophil or macrophage cell count in the lung after the administering.

In one embodiment, liposomes comprised of a vesicle-forming lipid derivatized with polyethylene glycol are provided. An exemplary derivatized lipid is distearoyl-polyetheylene glycol.

In another embodiment, liposomes having the therapeutic agent entrapped within the liposomes are provided. In another embodiment, the therapeutic agent is associated with external liposome surfaces. The therapeutic agent, in other embodiments, can be selected from the group consisting of anti-viral agents, anti-inflammatory agents, anti-bacterial agents, anti-fungal agents, gene therapy agents, and chemotherapeutic agents.

These and other objects and features of the invention will be more fully appreciated when the following detailed description of the invention is read in conjunction with the accompanying drawings.

### BRIEF DESCRIPTION OF THE DRAWINGS

FIGS. 1A-1B show the chemical structures of lipopolymers, mPEG-distearoyl (FIG. 1A) and mPEG-distearoylphosphatidylethanolamine (FIG. 1B);

FIG. 2 is a graph showing the spray particle size distribution of liposome particles, in micrometers, generated from four commercial nebulizers from Baxter Healthcare Corp. (Baxter 2083), Invacare Corporation (Sidestream®), Pari GmBH (Pari LC Plus®), and Aerogen, Inc. (AeroNeb®);

FIG. 3 shows the mass fraction of liposome formulation as a function of size, in μm, of liposome formulations aerosolized using a Pari LC Plus® nebulizer for liposome formulation nos. 1 (diamonds), 2 (x symbols), 3 (triangles), and 4 (squares);

FIG. 4 is a graph showing the percentage of ciprofloxacin released into a model lung surfactant (Survanta®), as a function of time, in hours, for liposome formulation nos. 1 (diamonds), 2 (x symbols), 3 (triangles), and 4 (squares);

FIG. 5 is a graph showing the ciprofloxacin uptake, in pg/cell, into macrophages as a function of time, in minutes, for free ciprofloxacin (inverted triangles) liposome formulation nos. 1 (diamonds), 2 (x symbols), 3 (triangles), and 4 (squares);

FIG. 6A is a graph showing the plasma concentration of ciprofloxacin, in ng/mL, as a function of time, in minutes, after intracheal administration to rats of free ciprofloxacin (inverted triangles) and of liposome formulation nos. 1 (diamonds), 2 (x symbols), 3 (triangles), and 4 (squares);

FIG. 6B is a graph showing the plasma concentration of ciprofloxacin, in ng/mL, as a function of time, in minutes, after intracheal administration to rats of free ciprofloxacin (inverted triangles) liposome formulation nos. 1 (diamonds), 5 (closed circles), and 6 (open circles);

FIGS. 7A-7B are bar graphs showing the concentration of ciprofloxacin in the lungs of rats 48 hours after intratracheal instillation of ciprofloxacin liposome formulation nos. 1-4 and of free ciprofloxacin, FIG. 7A and 7B differ only in the y-axis;

FIGS. 7C-7D are bar graphs showing the concentration of ciprofloxacin in the lungs of rats 48 hours after intratracheal instillation of ciprofloxacin liposome formulation nos. 1,6, and 7 and of free ciprofloxacin, FIG. 7C and 7D differ only in the y-axis; and

FIGS. 8A-8H are photomicrographs of cells recovered from bronchoalveolar lavages viewed under fluorescent microscopy, the lavages taken from mice after intranasal administration of phosphate buffered saline (FIGS. 8A-8B); a positive control, zymosan (FIGS. 8C-8D); conventional liposomes lacking a surface coating of PEG (FIGS. 8E-8F); and PEG-coated liposomes (FIGS. 8G-8H).

### DETAILED DESCRIPTION OF THE INVENTION

I.Definitions

As used herein, the term “aerosol” refers to dispersions in air of solid or liquid particles, of fine enough particle size and consequent low settling velocities to have relative airborne stability

“Liposome aerosols” consist of aqueous droplets within which are dispersed one or more particles of liposomes or liposomes containing one or more medications or diagnostic agents intended for delivery to the respiratory tract of man or animals. The size of the aerosol droplets are mass median aerodynamic diameter (MMAD) of 1-5 μm with a geometric standard deviation of about 1.5-2.5 μm.

The following abbreviations are used herein: PEG, poly(ethylene glycol); mPEG, methoxy-PEG; DSPE, distearoyl phosphatidylethanolamine; mPEG-DSPE, mPEG covalently linked to distearoylphosphatidylethanolamine; HSPC, hydrogenated soy phosphatidylcholine; mPEG-DS, mPEG covalently linked through a carbamate linkage to distearoyl; chol, cholesterol.

### Liposome Composition and Preparation

Liposomes are closed lipid vesicles used for a variety of therapeutic purposes, and in particular, for carrying therapeutic agents to a target region or cell by in vivo administration of liposomes. Liposomes are typically formed of vesicle-forming lipids, i.e., lipids that spontaneously form bilayer vesicles in water. The vesicle-forming lipids preferably have two hydrocarbon chains and a polar head group. There are a variety of synthetic vesicle-forming lipids and naturally-occurring vesicle-forming lipids known in the art where the two hydrocarbon chains are typically from about 12 to about 24 carbon atoms in length, and have varying degrees of unsaturation. Examples include the phospholipids, such as phosphatidylcholine (PC), phosphatidylethanolamine (PE), phosphatidic acid (PA), phosphatidylinositol (PI), and sphingomyelin (SM). A preferred lipid for use in the present invention is hydrogenated soy phosphatidylcholine (HSPC). Another preferred family of lipids are diacylglycerols. These lipids can be obtained commercially or prepared according to published methods.

The vesicle-forming lipid may be selected to achieve a degree of fluidity or rigidity, to control the stability of the liposome in serum, and to control the rate of release of an entrapped agent in the liposome. Liposomes having a more rigid lipid bilayer, or a liquid crystalline bilayer, can be prepared by incorporation of a relatively rigid lipid, e.g., a lipid having a relatively high phase transition temperature, e.g., up to about 80° C. Rigid lipids, i.e., saturated, contribute to greater membrane rigidity in the lipid bilayer. Other lipid components, such as cholesterol, are also known to contribute to membrane rigidity in lipid bilayer structures.

Lipid bilayer fluidity is achieved by incorporation of a lipid having a relatively low liquid to liquid-crystalline phase transition temperature, e.g., at or below room temperature (about 20-25° C.).

The liposome can also include other components that can be incorporated into lipid bilayers, such as sterols. These other components typically have a hydrophobic moiety in contact with the interior, hydrophobic region of the bilayer membrane, and a polar head group moiety oriented toward the exterior, polar surface of the membrane.

Another lipid component in the liposomes of the present invention, is a vesicle-forming lipid derivatized with a hydrophilic polymer. This lipopolymer component results in formation of a liposome surface coating with hydrophilic polymer chains on both the inner and outer lipid bilayer surfaces. Typically, between about 1-20 mole percent of the lipopolymer is included in the lipid composition. Liposomes having a surface coating of hydrophilic polymer chains, such as polyethylene glycol (PEG), are desirable as drug carries as these liposomes offer an extended blood circulation lifetime over liposomes lacking the polymer coating. The polymer acts as a barrier to blood proteins thereby preventing binding of the protein and recognition of the liposomes for uptake and removal by macrophages and other cells of the reticuloendothelial system.

Hydrophilic polymers suitable for derivatization with a vesicle-forming lipid include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, and polyaspartamide. The polymers may be employed as homopolymers or as block or random copolymers.

A preferred hydrophilic polymer chain is poly(ethyleneglycol) (PEG), preferably as a PEG chain having a molecular weight between about 500 to about 10,000 Daltons, preferably between about 1,000 to about 5,000 Daltons. Methoxy or ethoxy-capped analogues of PEG are also preferred hydrophilic polymers. These polymers are commercially available in a variety of polymer sizes, e.g., from about 12 to about 220,000 Daltons. A preferred lipopolymer is mPEG-DPSE.

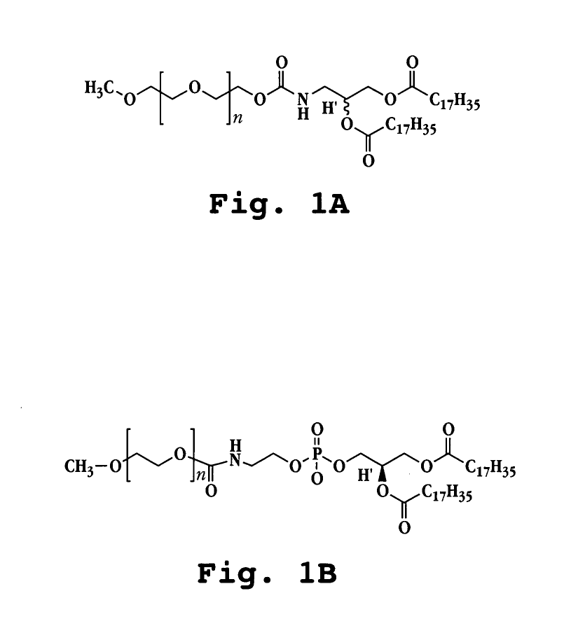

Another lipopolymer contemplated for use in the liposomes is the neutral lipopolymer described in U.S. Pat. No. 6,586,001 and referred to as mPEG-DS. The disclosure relating to preparation and characterization of this lipopolymer is incorporated by reference herein. FIG. 1A shows the structure of mPEG-DS. The hydrophilic polymer is linked to the hydrophobic portion, distearoyl, through a carbamate linkage. It will be appreciated that the hydrophobic portion can be selected from a wide range of hydrophobic species and that the C18 diacyl chains are merely exemplary. Alternative hydrophobic species are described in U.S. Pat. No. 6,586,001. It will also be appreciated that the carbamate linkage is merely exemplary, and other linkages are apparent to a skilled chemist. For comparison, the structure of mPEG-DSPE is shown in FIG. 1B, where the polymer is linked to the hydrophobic species at the phosphatidyl head group.

The liposomes can optionally contain a targeting ligand, as are widely known in the art.

The liposomes include a therapeutic agent or a diagnostic agent and it will be appreciated that the agent can be entrapped in the liposomes or associated with the external liposome surface, such as by tethering the agent to a lipid or to a hydrophilic polymer. Any therapeutic or diagnostic agent is suitable, and those of skill in the art can easily select an agent for treatment of a certain disease or condition.

The liposomal composition described herein is intended for administration via inhalation. For inhalation therapy, the delivery is achieved by (a) aerosolization of a dilute aqueous suspension by means of a pneumatic nebulizer, (b) spraying from a self-contained atomizer using a propellant solvent with suspended, dried liposomes in a powder, (c) spraying dried particles into the lungs with a propellant or (d) delivering dried liposomes as a powder aerosol using a suitable device.

Pulmonary Delivery

In studies conducted in support of this invention, six liposomal formulations were prepared for analysis. Preparation of the formulations is set forth in Example 1 and the components and method of drug loading is Table 1.

TABLE 1
Formulation Compositions and Method of Drug Loading

| Formulation | | |
|---|---|---|
| No. | | Cirprofloxacin Loading |
| (Abbreviation) | Lipid Composition | Method |
| 1 | HSPC/chol/mPEG-DSPE | remote loading against |
| (PEG-AS) | (50/45/5) | ammonium sulfate |
| | | gradient |
| 2 | HSPC/chol/mPEG-DSPE | remote loading against |
| (PEG-DAS) | (50/45/5) | dextran ammonium sulfate |
| | | gradient |
| 3 | HSPC/chol/mPEG-DSPE | passive entrapment |
| (PEG-PE) | (50/45/5) | |
| 4 | HSPC/chol | remote loading against |
| (C-AS) | (55/45) | ammonium sulfate |
| | | gradient |
| 5 | PHSPC:chol:mPEG | remote loading against |
| (PHSPC) | (50/45/5) | ammonium sulfate |
| | | gradient |
| 6 | egg phosphatidyl | remote loading against |
| (egg PC) | choline:chol:mPEG | ammonium sulfate |
| | (50/45/5) | gradient |

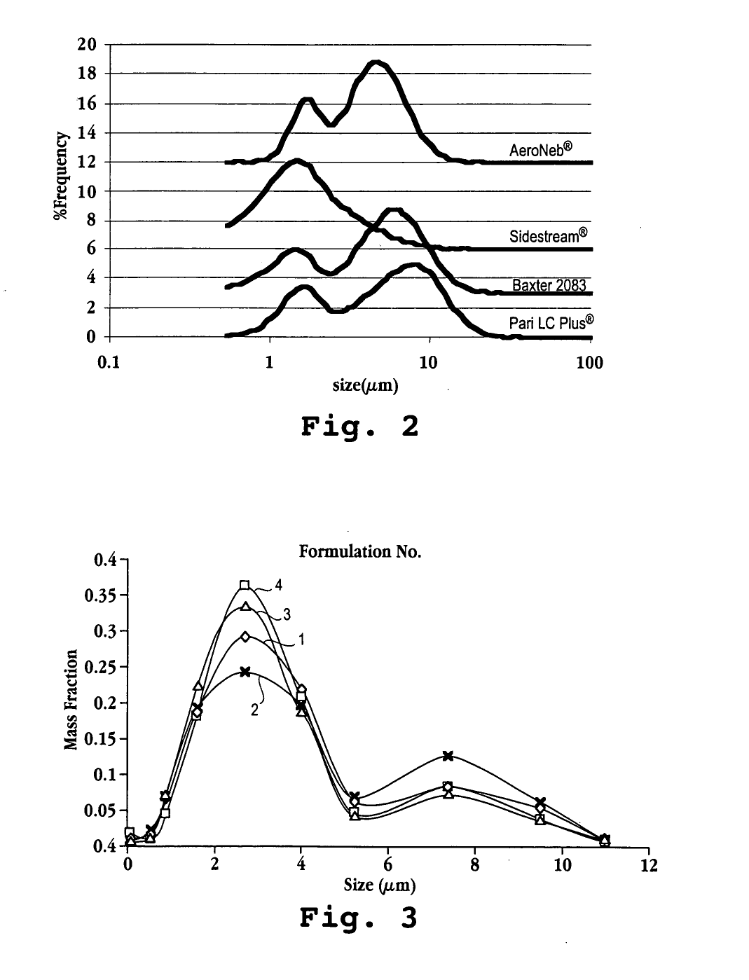

Formulation No. 1 was neublized in four commercially-available nebulizer from Baxter Healthcare Corp. (Baxter 2083), Invacare Corporation (Sidestream®), Pari GmBH (Pari LC Plus®), and Aerogen, Inc. (AeroNeb®). As describe in Example 2, a defined volume of the liposomal-ciprofloxacin formulation no. 1 into each nebulizer and aerosolized according to the manufacturer's instructions. The particle size and distribution were evaluated using a Malvern Mastersizer based on Frauenhofer Diffraction Pattern Analysis. The aerosol particle distribution of the liposomes generated by the four nebulizer is shown in FIG. 2.

A unique distribution pattern of particle size was generated by each nebulizer. Distribution profiles of geometric mean diameters were similar to drug in water, in the absence of liposomes or any other salts in solution. The Pari, Baxter and AeroNeb nebulizers despite having very disparate mechanisms exhibited similar bimodal distributions of geometric mean diameters. Interestingly, the SideStream demonstrated a unimodal distribution with the majority of the particles having a geometric mean diameter <5 μm.

The mean mass diameters (D50 μm) of particles generated for Formulations from each nebulizer are summarized in Table 2.

TABLE 2
Summary of Mean Mass Diameters of Particles
Generated from Different Nebulizers

| Formulation | Mass Mean Diameter (μm) | | | |
|---|---|---|---|---|
| No. | for Indicated Nebulizer Type | | | |
| (Abbre- | | Pari LC | | |
| viation) | SideStream ® | Plus ® | Baxter 2083 | AeroNeb ® |
| 1 | 1.48 ± 0.01 | 5.0 ± 0.2 | 4.6 ± 0.1 | 4.0 ± 0.1 |
| (PEG-AS) | | | | |
| 2 | 1.45 ± 0.05 | 4.2 ± 0.4 | 4.66 ± 0.05 | 3.6 ± 0.2 |
| (PEG-DAS) | | | | |
| 3 | 1.40 ± 0.03 | 4.4 ± 0.4 | 4.3 ± 0.7 | 4.5 ± 0.2 |
| (PEG-PE) | | | | |
| 4 | 1.76 ± 0.01 | 4.04 ± 0.05 | 5.12 ± 0.05 | 3.7 ± 0.1 |
| (C-AS) | | | | |

Table 2 shows that mean diameters were equivalent for all formulations of liposomal drug for each of the four conventional nebulizers evaluated. The emitted aerosol size from the nebulizers was dependent on nebulizer mechanism rather than liposomal formulation. There was a significant difference in the mean aerodynamic size of particles emitted from the SideStream® nebulizer, whereas the aerodynamic diameters were similar for the other nebulizers assessed, including the Baxter 2083, Pari LC Plus®, and AeroNeb®. This is despite the vastly different nebulizer mechanism for the AeroNeb®. The AeroNeb® uses a piezo-electric vibrational plate to pump liquid through a mesh. The mass median diameter is well within the respirable range for deposition of aerosol particles into the deep lung. Therefore, aerosolization of liposomal drug generated by conventional nebulization was capable of generating the appropriate-sized aerosol particles for deposition into the lung. Respirable fractions (1-5 μm) particles could be generated using from the liposomal ciprofloxacin formulations and in aerodynamic diameter suitable for use.

In another study, described in Example 3, the influence of formulation composition on nebulisate output was examined. Formulation nos. 1-4 (Table 1 above) were placed into the Pari LC Plus® nebulizer and aerosolized until dryness, with the spray collected on an impactor plate. The aerosol particles were recovered by washing and the distribution analyzed. The results are shown in FIG. 3, for liposome formulation nos. 1 (diamonds), 2 (x symbols), 3 (triangles), and 4 (squares). The distribution of aerosol particles onto the plates was similar for each nebulizer, with a bimodal distribution of aerosol particles with a larger number of particles and larger total mass at a smaller aerodynamic particle size was observed for each formulation.

In administering an agent to the lung in the form of a liposomal carrier, it is desirable that the liposome particle remain intact after aerosolization. This is particularly desirable to achieve a depot reservoir of drug for release over an extended period of time. Example 4 describes a study to determine liposome intactness and extent of drug leakage after nebulization. Formulation nos. 1-4 (see Table 1) were aerosolized using the Pari LC Plus® nebulizer and the nebulisate was collected into a flask. After removal of any ciprofloxacin unentrapped within a liposome by dialysis, aliquots of the nebusilate were lysed and analyzed for ciprofloxacin concentration. As a control, the ciprofloxacin concentration in liposomes not subjected to nebulization was determined. The results are summarized in Table 4A as the percent ciprofloxacin entrapped in liposomes of each formulation after nebulization, relative to a non-nebulized sample of the same formulation.

TABLE 4A
Percent of Ciprofloxacin Entrapped in Liposomes
Before and After Nebulization

| | Formulation No.¹ | | | |
|---|---|---|---|---|
| | (Abbreviation) | | | |
| | 1 | 2 | 3 | 4 |
| | (PEG-AS) | (PEG-DAS) | (PEG-PE) | (C-AS) |
| Percent Drug Entrapped | 96 | 85 | 62 | 96 |
| in Liposome Before | | | | |
| Nebulization | | | | |
| Percent Drug Entrapped | 78 | 73 | 40 | 48 |
| in Liposome After | | | | |
| Nebulization | | | | |
| percent loss due to | 19 | 14 | 36 | 50 |
| nebulization | | | | |

¹see Table 1 for the composition of each formulation.

A similar study was conducted using a single liposomal formulation, Formulation no. 1, nebulized by the Pari LC Plus®, Baxter 2083, and AeroNeb® units. Liposome intactness after nebulization was evaluated as described in Example 4 and the results are summarized in Table 4B.

TABLE 4B
Percent of Ciprofloxacin Entrapped in Liposomes of Formulation
No. 1 Before and After Nebulization from various Nebulizers

| | Nebulizer | | |
|---|---|---|---|
| | Baxter | Pan LC | |
| | 2083 | Pluse ® | AeroNeb ® |
| Percent Drug Entrapped in | 96 | 96 | 96 |
| Liposome Before Nebulization | | | |
| Percent Drug Entrapped in | 68 | 78 | 44 |
| Liposome After Nebulization | | | |
| percent loss due to nebulization | 29 | 19 | 54 |

¹see Table 1 for the composition of formulation no. 1

The amount of ciprofloxacin remaining encapsulated within the liposome was highest for the Pari LC Plus® nebulizer with 78% ciprofloxacin remaining encapsulated after nebulization, from a starting percent encapsulation of 96%. The nebulisate from the Baxter 2083 nebulizer resulted in 68% ciprofloxacin encapsulated, while the AeroNeb® nebulizer destabilized the liposomes as evidenced by the 54% loss of entrapped drug due to nebulization. The nebulizer mechanism that resulted in the least degradation was the conventional jet nebulizer mechanism whereby a stream of compressed air draws liquid into the air and causes spontaneous formation of the aerosol particles as a result of surface tension between the air and water. Nebulizers with an ultrasonic vibrational mechanism to generated aerosol particles appear to be least likely to destabilize the liposomes.

In another study, release of ciprofloxacin from the liposomal formulations into a model lung surfactant (Survanta®) was determined as a function of time over a 48 hour test period. As described in Example 5, each formulation (Formulation nos. 1-4, Table 1) were combined with Survanta® and dialyzed against a phosphate buffer. Samples were removed periodically for analysis of ciprofloxacin concentration, and the results are shown in FIG. 4. Formulation no. 3 (triangles) in which ciprofloxacin was passively entrapped afforded the highest rate of release, with about 60% of the drug released at the 24 hour time point. The two formulations where ciprofloxacin was remotely loaded into the liposomes against and ammonium sulfate gradient, formulation no. 1 (diamonds) and formulation no. 4 (squares), had the slowest rate of release, with less than 10% of the entrapped drug released into the medium at the 24 hour time point. Formulation nos. 2 (x symbols) was intermediate in its release rate relative to the other formulations.

This data illustrates that the use of the ammonium sulfate gradient was able to prevent immediate release of drug from the liposome interior. Formulations which were generated using an ammonium sulfate gradient demonstrated at least a 50-800% increase in the amount of ciprofloxacin remaining encapsulated inside the liposome interior when compared to the passively encapsulated formulation (formulation no. 3). A change in the pH of the lung fluid caused negligible differences in the release rate of the ciprofloxacin from the conventional liposome formulation (data not shown). Formulation no. 2, with dextran ammonium sulfate, did not provide an improved stability beyond that of ammonium sulfate alone and may have caused a decrease in stability. The higher release rate with formulation no. 2 may also be due to the presence of dextran-ammonium sulfate-ciprofloxacin complexes on the exterior of the liposome that were not removed during the liposomal preparation process. Whether the drug was encapsulated within a conventional (non-PEG, formulation no. 4) or pegylated (formulation no. 1) liposome did not confer different stability or release of ciprofloxacin into the media.

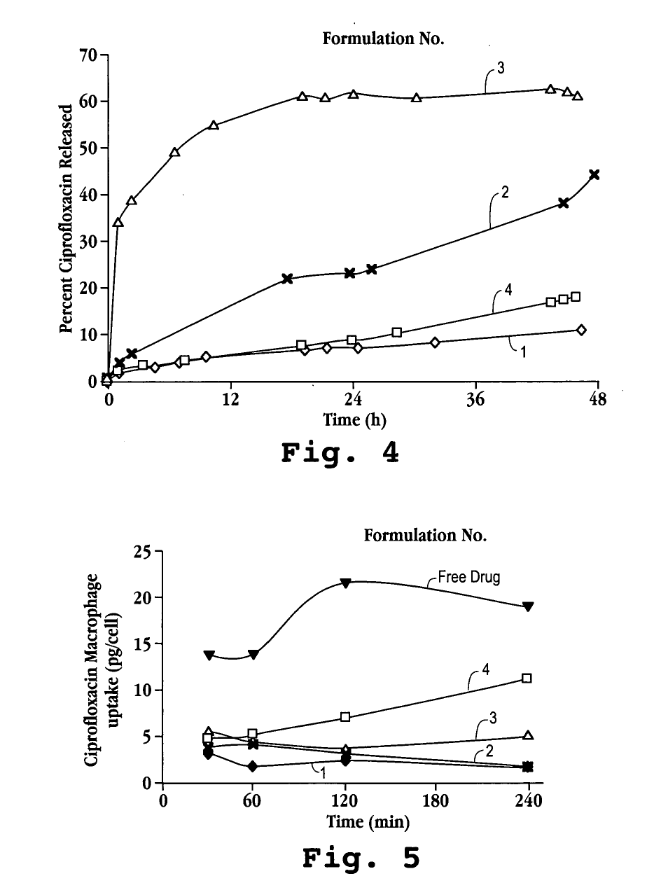

One of the key components in an inflammatory response in the lung is the activation of macrophages, where resident macrophages are a first line of cellular defense. An in vitro study was conducted to evaluate the extent of macrophage uptake of liposome formulation nos. 1-4 and of free ciprofloxacin. As described in Example 6, rat alveolar macrophages were grown in culture. Cells were placed in a test tube along with liposome formulation no. 1, 2, 3, or 4, or with free ciprofloxacin, at a drug concentration of 0.5 mg/mL. The cells were incubated in the presence of the formulation for 4 hours at 37° C., and aliquots of the cells were removed for determination of ciprofloxacin uptake. The results are shown in FIG. 5, where the ciprofloxacin uptake, in pg/cell, into the macrophage cells as a function of time, in minutes, for free ciprofloxacin (inverted triangles) liposome formulation nos. 1 (diamonds), 2 (x symbols), 3 (triangles), and 4 (squares) is graphed.

The data shows that ciprofloxacin administered to the cells in the form of a liposomal carrier reduces uptake of the drug by the macrophages. The uptake of free ciprofloxacin (inverted triangles) was higher than for any of the liposomal formulations. Liposome formulations having a coating of polyethylene glycol (PEG) (formulation nos. 1 (diamonds), 2 (x symbols), 3 (triangles) had a reduced alveolar macrophage uptake when compared to conventional, non-peg-coated liposomes (formulation no. 4, squares). The combination of an ammonium sulfate gradient and a steric barrier offered by the PEG coating reduced alveolar macrophage uptake, along with the advantage of minimal leakage of the drug into the alveolar space. Thus, liposome formulations having an ion gradient and a surface coating of hydrophilic polymer chains, as exemplified by an ammonium sulfate gradient and a coating of PEG, offer a sustained release delivery system for the lung.

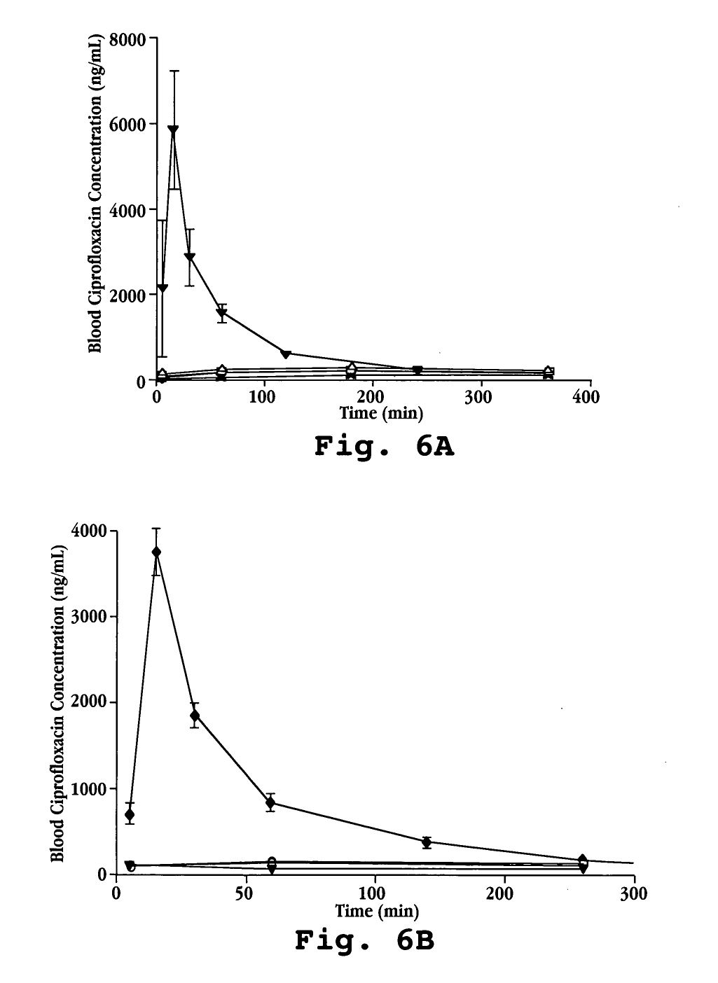

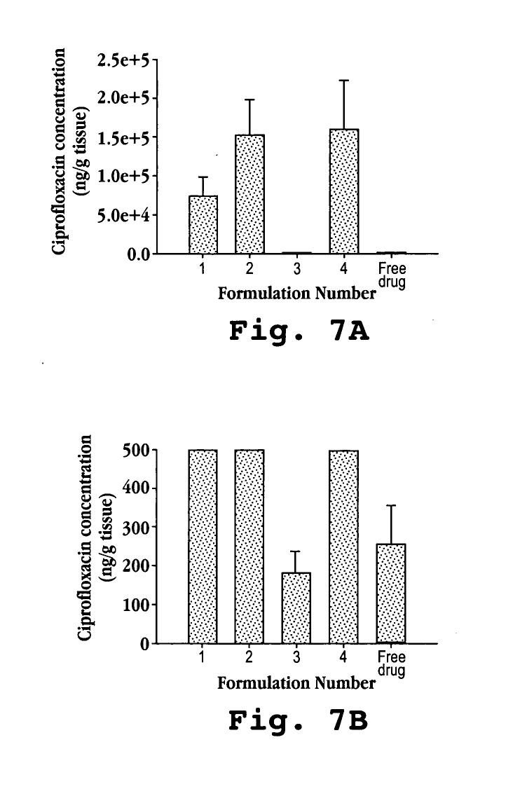

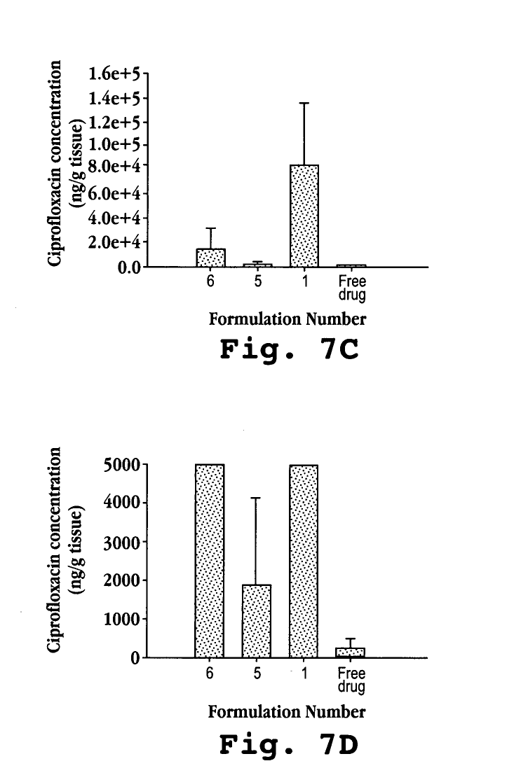

An in vivo study was performed, where liposome formulation nos. 1-6 were administered to the lungs of rats via tracheal infusion using a catheter. As described in Example 7, after infusion of the formulation, blood samples were taken and analyzed for ciprofloxacin concentration. Forty-eight hours after administration, the lungs were removed and the ciprofloxacin concentration in the lung tissue was quantified. The results are shown in FIGS. 6A-6B and 7A-7D.

FIG. 6A is a graph showing the blood concentration of ciprofloxacin, in ng/mL, released from the liposome formulations as a function of time, in minutes. As a comparative control, free ciprofloxacin (inverted triangles) was administered intracheally and its concentration in the plasma analyzed. Free ciprofloxacin (inverted triangles) when administered to the lungs results in a significant and detectable amount of drug in the blood shortly after administration. Ciprofloxacin entrapped in liposome formulation nos. 1 (diamonds), 2 (x symbols), 3 (triangles), and 4 (squares) is released slowly, if at all, into the blood compartment after tracheal infusion.

FIG. 6B shows the results for liposome formulation nos. 5 (closed circles) and 6 (open circles) (see Table 1, above) along with liposome formulation no. 1 (diamonds) and free ciprofloxacin (inverted triangles) for comparison. The three liposomal formulations, nos. 1, 5, and 6, provided a slow, minimal release of drug into the blood after in vivo tracheal administration, indicating the suitability of the liposomal carrier as a drug reservoir depot.

The ciprofloxacin concentration in the lungs of the test animals, harvested 48 hours after tracheal infusion of the liposomal formulations, is shown in FIGS. 7A-7D. FIGS. 7A-7B are bar graphs showing the concentration of ciprofloxacin in the lungs of rats 48 hours after intratracheal instillation of ciprofloxacin liposome formulation nos. 1-4 and of free ciprofloxacin. FIG. 7A and 7B differ only in the y-axis scale, with FIG. 7B having a smaller scale of 0-600 ng/g tissue for visibility of the concentration in the lungs from formulation no. 3 and from free ciprofloxacin. FIGS. 7C-7D are bar graphs showing the concentration of ciprofloxacin in the lungs of rats 48 hours after intratracheal instillation of ciprofloxacin liposome formulation nos. 5-6 and of free ciprofloxacin, with FIG. 7D showing the data presented on a y-axis scale of 0-600 ng/g tissue. The data in FIGS. 7A-7D show that a low amount of ciprofloxacin was recovered in the lung tissue when the drug is administered in free form, from liposomes in which the drug was entrapped passively (formulation no. 3), or when the liposome is comprised of primarily lipids in the fluid phase at 37° C., as in formulation nos. 5 and 6. In contrast, delivery of ciprofloxacin from liposomes formed of relatively rigid lipids and when the drug is loaded into the liposomes against an ion gradient, a depot of drug in the lungs is provided, for sustained release of the drug.

In another in vivo study, described in Example 8, liposomes having a surface coating of PEG and conventional liposomes with no surface coating of PEG were administered to mice intranasally. As a positive control, zymosan, an insoluble preparation of yeast cells known to activate macrophages via toll-like receptor 2, was intranasally administered. Another group of control mice were treated with phosphate buffered saline intranasally. Six hours after administration, bronchoalveolar lavages were taken and quantified for inflammatory cell infiltration of neutrophils and macrophages. The cell activation upon intranasal administration was quantitated using cell counts of neutrophils and macrophages and the counts are shown in Table 5.

TABLE 5
Bronchoalveolar Lavage Cell Counts of Inflammatory
Cell Infiltration Six Hours after Intranasal Administration
of Liposomes, Zymosan, or Saline

| | Average Cell Count (×10⁴) ± SD | | |
|---|---|---|---|
| | | | Total Cell Count |
| | | | (sum of macrophage |
| Group | Macrophages | Neutrophils | and neutrophils) |
| Control, saline | 1.7 ± 1.3 | 1.79 ± 1.3 | 3.5 ± 2.7 |
| Zymosan | 6.8 ± 4.8 | 24.4 ± 14.3 | 32.3 ± 18.7 |
| Conventional | 4.8 ± 2.8 | 7.4 ± 7.9 | 12.1 ± 10.2 |
| Liposomes (no PEG)¹ | | | |
| PEG-coated | 2.4 ± 1.2 | 1.3 ± 0.9 | 3.8 ± 1.4 |
| liposomes¹ | | | |

¹see Example 8 for the composition of each formulation.

The data in Table 5 shows that PEG-coated liposomes did not induce an inflammatory response, as evidenced by no observable difference in the cell number and type recovered in the bronchoalveolar lavages from control animals treated with saline and animals treated with PEG-coated liposomes. Intranasal administration of zymosan caused a significant influx of cells into the airway, as expected. Administration of conventional liposomes lacking a coating of PEG, and which contain 20 mole % negative charge induced an inflammatory response, as evidenced by the elevated neutrophil and macrophage cell counts relative to the animals treated with saline. The data, in summary, clearly establishes that no inflammatory response was observed due to the presence of PEG-coated liposomes in the airways.

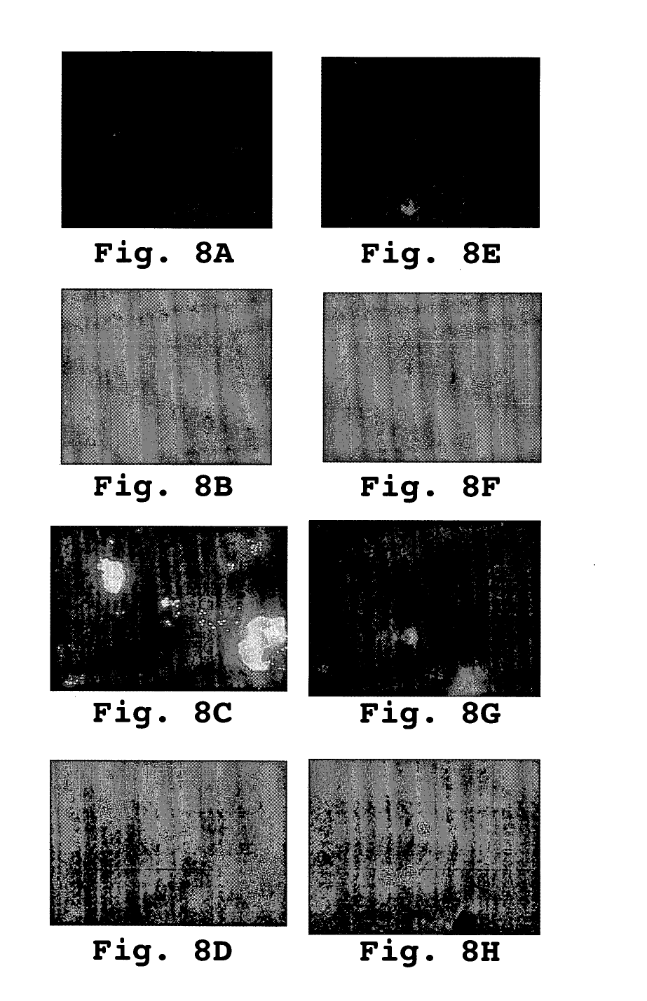

The photomicrographs of the bronchoalveolar lavages viewed under fluorescent microscopy are shown in FIGS. 8A-8H. FIGS. 8A-8B correspond to the bronchoalveolar ravages of mice treated with phosphate buffered saline; FIGS. 8C-8D correspond to bronchoalveolar lavages of mice treated with the positive control zymosan; FIGS. 8E-8F correspond to bronchoalveolar ravages of mice treated with conventional liposomes lacking a surface coating of PEG; and FIGS. 8G-8H correspond to bronchoalveolar ravages of mice treated with PEG-coated liposomes. For all photomicrographs, there is some autofluoresence of the macrophages upon viewing under fluorescence conditions. As seen in FIGS. 8C-8D, intranasal administration of zymosan resulted in uptake of the zymosan by the cells, evidenced by the punctuated structures which are indicative of the presence of intracellular endocytotic bodies. Both types of liposomes, PEG-coated and conventional, non-PEG coated, appeared to have been associated or internalized by the macrophages. The fluorescence of the conventional liposomes (FIGS. 8E-8F) is more granular in nature compared to that of the PEG-coated liposomes (FIGS. 8G-8H) suggesting cellular uptake by conventional liposomes compared to cell surface association by PEG-coated liposomes.

From the foregoing, various aspects and features of the invention can be appreciated. Delivery systems or drugs that bear a charge can cause inflammatory reactions by inducing macrophage uptake and subsequent neutrophil infiltration to the pulmonary area. Highly charged drug delivery systems will be particularly efficient in inducing inflammatory or immune effects in the lung which can cause compromised lung function. For example cationic lipids cause inflammatory effect by inducing cytokine production and reactive oxygen intermediates (Dokka, S., et al., Pharm. Res., 18(5):521 (2000)). Negative charges in a delivery system have also been shown to cause complement activation (Cunningham, C. M. et al., J. Immunol., 122(4):1238 (1989)). Drugs and molecules that are not highly charge may still have the propensity to induce an inflammatory effect in the absence of a carrier. The studies herein establish that encapsulation of drugs inside liposomes, which do not contain immune stimulatory molecules and which have a protective barrier against an immune response, are able to reduce induction of an immune response. In particular, liposomes which include the features of (i) a hydrophilic polymer coating on the external liposome surface decreases the potential for charge effects by shielding the liposome and the entrapped drug from binding with proteins, cell membranes, etc. and from interaction with receptors on cell surfaces; (ii) an ion gradient, such as an ammonium sulfate gradient or pH gradient, retains the drug in the liposome providing for a sustained drug release and reduced inflammatory reaction.

### V.EXAMPLES

The following examples further illustrate the invention described herein and are in no way intended to limit the scope of the invention.

Materials

All materials were obtained from commercially suitable vendors, such as Aldrich Corporation.

### Example 1

### Preparation of Liposomes Containing Ciprofloxacin

HSPC, cholesterol and, in some formulations, mPEG-DSPE were solubilized in ethanol. Multilamellar vesicles were formed using the ethanol injection technique where the ethanol solution of lipids were hydrated in ammonium sulfate at pH 5.5 and at 65° C. Liposomes were downsized to ˜150 nm by extrusion through an extruder at 65° C. using serial size decreasing membranes —0.4 μm, 0.2 μm and 0.1 μm. External ammonium sulfate was removed by exchanging against 10% sucrose, NaCl (pH=5.5) using diafiltration to generate an ion gradient. Ciprofloxacin was solubilized in 10% sucrose and incubated with the liposomes at 65° C. for 30-60 min. Free ciprofloxacin was removed using diafiltration against 10% sucrose, NaCl. Typical loading resulted in 40-60% of initial drug concentration loaded into liposomes. The final solution was in a 10 mM histidine and 10% sucrose buffer. Typical drug to lipid ratios were 0.3-0.5 (w/w).

Liposomes were also prepared using a passive encapsulation procedure. The lipids HSPC, cholesterol, and mPEG-DSPE were solubilized in ethanol. The solubilized lipids were added to a high concentration of ciprofloxacin solution (120 mg/mL) at 65° C. for 60 minutes. Liposomes were then downsized to ˜150 nm by extrusion at 65° C. through 0.4 μm, 0.2 μm and 0.1 μm size membranes. Unencapsulated ciprofloxacin was removed using diafiltration against 10% sucrose, NaCI (pH=5.5) and 10% sucrose, 10 mM histidine (pH=6.5). Typical loading resulted in drug to lipid ratios of at least 0.3 (w/w).

The formulations prepared are summarized in Table 1.

### Example 2

### Aerosol Particle Formation of Liposomes

Liposomes were prepared containing ciprofloxacin according to Example 1. A measured volume (2-3 mL) of each liposomal ciprofloxacin formulation was placed in a reservoir of a nebulizer. Four commercially-available nebulizers (Baxter Healthcare Corp. (Baxter 2083), Invacare Corporation (Sidestream®), Pari GmBH (Pari LC Plus®), and Aerogen, Inc. (AeroNeb®)) were obtained and used to aerosolize the liposomal ciprofloxacin formulations. The aerosolized particle size and distribution were evaluated using a Malvern Mastersizer based on Fraunhofer Diffraction Pattern Analysis. During the aerosolization process, the nebulizer was aligned so that the spray passed through the analysis beam of the Fraunhofer instrument, at the designated sample plane for the device, with care taken to maintain the sample place since deviations from this sample plane will cause vignetting of the scattering pattern and incorrect size distribution results. Approximately one minute of nebulization was initially performed before placing into the analysis beam in order to avoid startup effects from affecting the size distribution measurement. After this initial period, the nebulizate spray was analyzed with the scattering pattern collected for 30 seconds. The Mastersizer software was used to calculate the spray particle size distribution and associated statistical measures on a mass basis (D_{3,2}, D₅₀, D₉₀,). The results are shown in FIG. 2.

### Example 3

### Aerosol Particle Formation of Liposomes

A known amount of liposomal ciprofloxacin was placed into the reservoir of the nebulizer. Nebulization of the liquid formulation proceeded into an Andersen cascade impactor until no further aerosolization occurred; i.e. run to dryness. The plates were washed with buffer to collect the sample deposited. The buffer was comprised of 10 mM sodium phosphate monobasic dihydrate, 140 mM saline and 10% methanol at pH 3.5. The concentration of ciprofloxacin deposited on various plates of the cascade impactor was determined using UV spectrophotometry analysis. The results are shown in FIG. 3.

### Example 4

### Analysis of Liposome Stability After Aerosolization

Liposomal ciprofloxacin formulations prepared as described in Example 1 were aerosolized using the Pari LC Plus® nebulizer and the nebulisate was collected into a Erlenmeyer flask containing PBS buffer. The collected nebulisate was dialyzed overnight against at least 50× PBS buffer (pH =3.8) to remove unencapsulated ciprofloxacin. For controls, methanol was added to the nebulisate to a final concentration of 10% methanol and also dialyzed overnight against PBS buffer. UV spectrophotometry at absorbance=288 nm was used to assay for ciprofloxacin in the dialysis buffer and in lysed aliquots of the nebulisate in the dialysis bag. A comparison of the encapsulation fraction between the nebulized and non-nebulized liposomes was made. The results are shown in Tables 4A-4B.

### Example 5

### In vitro Release of Ciprofloxacin in Model Lung Surfactant

Liposome formulations prepared according to Example 1. Each formulation was combined with Survanta®, a modified natural bovine lung extract (Ross Products Division, Abbott Laboratories, Inc., Columbus Ohio) at a ratio of 1:5 and placed into dialysis tubing. Each formulation and Survanta® was dialyzed against phosphate buffer (pH=3.5) over 48 hours. Aliquots of 2 mL were removed from the external phase at 2-3 hour intervals. The results are shown in FIG. 4.

### Example 6

### In vitro Incubation of Liposomes and Rat Alveolar Macrophages

NR8383 cell lines (ATCC) were established to provide a homogeneous and continuous source of responsive alveolar macrophages to study macrophage-related activity. NR8383 was obtained from ATCC as a continuous culture of rat alveolar macrophages. The original culture was obtained from bronchoalveolar lavages from female Sprague-Dawley rats. NR8383 cells exhibited the following activities associated with macrophage activation: phagocytosis of zymosan, non-specific esterase activity, oxidative burst, F_{c}. receptors, and secretion of IL-1, TNFβ, and IL-6. The continuous cell line was subcultured in Ham's F12 media containing 15% FBS (Gibco), 2 mM L-Glutamine (Gibco) and 100 U/100 μg Penicillin/streptomycin (Sigma).

NR8383 cells growing in log-phase were prepared in Ham's F12 media in the absence of serum at a concentration of 1×10⁶ cells/mL. Cells were placed into 12×75 mm polypropylene test tubes along with a liposomal ciprofloxacin formulation (see Example 1, Table 1) or free ciprofloxacin at a drug (ciprofloxacin, Uquifa) concentration of 0.5 mg/mL. The lipid concentration ranged between 0.15-0.25 mg/mL total lipid. Cells were incubated for 4 hours at 37° C. and 5% CO₂ with each tube lying on the side to maximize surface area. Aliquots (200 μL) of cells were removed at timepoints 30 min, 70 min, 120 min, 240 min post-addition of liposomal drug. Removed aliquots were centrifuged at 200 g for 2 min and the supernatant removed. Pellets were washed two times using 30 mM sodium acetate/150 mM sodium chloride pH 4.5. Pellets recovered after the second washing were frozen at −70° C. overnight.

Frozen pellets were warmed to room temperature and assayed for cell number by CyQuant-GR probe (Molecular Probes, Eugene, OR) and ciprofloxacin by fluorometry. Pellets were resuspended in a solution containing lysis buffer to disrupt the cell membrane and CyQuant Green dye. Upon DNA binding, CyQuantGR dye emits at λ=520 nm at a excitation λ=480 nm. Ciprofloxacin emits at λ=450 nm at an excitation λ=350 nm. Cell number and ciprofloxacin concentration were interpolated from a standard curve containing cell number and CyQuantGR dye or ciprofloxacin. The results are shown in FIG. 5.

### Example 7

### In vivo Delivery of Liposomal Ciprofloxacin via Tracheal Infusion

A catheter was placed into the trachea of anesthetized rats and various formulations (prepared as described in Example 1, Table 1) of liposomal ciprofloxacin and free ciprofloxacin were then administered via catheter. Post-administration, blood was taken and ciprofloxacin concentration was determined in plasma using HPLC. The lung was removed after 48 hours and ciprofloxacin was extracted from the lung and assayed for concentration using HPLC-MS. The ciprofloxacin release rates for Formulation nos. 1-6 and for free ciprofloxacin are shown in FIGS. 6A-6B. The ciprofloxacin concentration in the lungs after removal is shown in FIGS. 7A-7D.

### Example 8

### Intranasal Administration of Liposomes to Mice

Liposomes having a coating of PEG were prepared from HSPC:chol:mPEG-DSPE:FITC-DHPE (55:40:5:01), (FITC=Fluorescein isothiocyanate; DHPE=dihexadecanoly-sn-glycerol-3-phosphoethanolamine). Conventional liposomes with no PEG coating were prepared from eggPC:DPPG:chol:FITC-DHPE (40:20:40:0.1).

Liposomes, positive zymosan control (FITC labeled), or phosphate buffered saline (PBS) control were administered to naive Balb/c mice via intranasal administration. Bronchoalveolar lavages using 1 mL PBS were performed at 6 hours post-administration. The recovery volume of each lavage was approximately 0.8 mL. The bronchoalveolar lavages were centrifuged at 1200 rpm for 10 minutes and supernatants removed. Cell pellets were resuspended and washed once more in PBS with 0.1% BSA. Cytospins were prepared and total cell number was determined by counting using a hemocytometer or fluorescence was determined by fluorescent microscopy. The results are shown in Table 6 and in FIGS. 8A-8H.

Although the invention has been described with respect to particular embodiments, it will be apparent to those skilled in the art that various changes and modifications can be made without departing from the invention.

1. A method for administering a therapeutic or diagnostic agent to a subject, comprising
providing a suspension of liposomes comprised of one or more of vesicle-forming lipids selected from (i) a vesicle-forming lipid derivatized with a hydrophilic polymer and (ii) a neutral lipopolymer, said liposomes being associated with said therapeutic or diagnostic agent;
forming an aerosol of said liposome suspension; and
administering said aerosol to said subject by inhalation, whereby said administering delivers intact liposomal particles to the respiratory tract of said subject to form a depot of therapeutic agent therein with no observable provocation of an immune response as measured by neutrophil or macrophage cell count in the lung after said administering.

2. The method of claim 1, wherein said providing includes providing liposomes comprised of a vesicle-forming lipid derivatized with polyethylene glycol.

3. The method of claim 1, wherein said providing includes providing liposomes comprised of distearoyl-polyetheylene glycol.

4. The method of claim 1, wherein said providing includes providing liposomes having a therapeutic agent entrapped within the liposomes.

5. The method of claim 1, wherein said providing includes providing liposomes having a therapeutic agent associated with external liposome surfaces.

6. The method of claim 4, wherein said providing includes providing liposomes having an entrapped therapeutic agent selected from the group consisting of anti-viral agents, anti-inflammatory agents, anti-bacterial agents, anti-fungal agents, gene therapy agents, and chemotherapeutic agents.

7. The method of claim 1, wherein said providing includes providing liposomes having a diagnostic agent associated with said liposomes.
